(19) United States
(12) Patent Application Publication
(10) Pub. No.: US 2024/0245466 A1
(43) Pub. Date: Jul. 25, 2024

(54) IDENTIFICATION OF GUIDEWIRE POSITION DURING A PROCEDURE

(71) Applicant: Libra Science Ltd., Road Town (VG)

(72) Inventor: Shlomo BEN-HAIM, Milan (IT)

(73) Assignee: Libra Science Ltd., Road Town (VG)

(21) Appl. No.: 18/399,774

(22) Filed: Dec. 29, 2023

Related U.S. Application Data
(60) Provisional application No. 63/440,721, filed on Jan. 24, 2023.

(51) Int. Cl. A61B 34/20; G06T 7/00

(52) U.S. Cl. CPC A61B 34/20; G06T 7/0012; A61B 2034/2055; G06T 2207/30021; G06T 2207/30048

(57) ABSTRACT

There is provided a computer implemented method of monitoring a guidewire position during a medical procedure, comprising: analyzing a plurality of baseline images captured over a plurality of at least one of heartbeats and breathing cycles, the plurality of baseline images depicting a guidewire in a body cavity, computing according to the analysis, a baseline movement of the guidewire during the at least one of heartbeats and breathing cycles, and monitoring successive images of the guidewire for detecting movement of the guidewire that deviates from the baseline movement.

### RELATED APPLICATION

This application claims the benefit of priority of U.S. Provisional Patent Application No. 63/440,721 filed on Jan. 24, 2023, the contents of which are incorporated herein by reference in their entirety.

### FIELD AND BACKGROUND OF THE INVENTION

The present invention, in some embodiments thereof, relates to image processing and, more specifically, but not exclusively, to systems and methods for analyzing medical images.

Guidewire are threaded through blood vessels of the body, for reaching a target anatomical location. Once the guidewire is in place, larger catheters and/or sheathes may be threaded over the guidewire, for example, for delivering devices to the target anatomical location (e.g., transcatheter valves, devices for closure of patent foramen ovale (PFO)) and/or for guiding treatment devices for applying treatment to the target anatomical location (e.g., radiofrequency ablation (RF), ultrasound).

### SUMMARY OF THE INVENTION

According to some embodiments of the present invention, there is provided a computer implemented method of monitoring a guidewire position during a medical procedure, comprising: analyzing a plurality of baseline images captured over a plurality of at least one of heartbeats and breathing cycles, the plurality of baseline images depicting a guidewire in a body cavity, computing according to the analysis, a baseline movement of the guidewire during the at least one of heartbeats and breathing cycles, and monitoring successive images of the guidewire for detecting movement of the guidewire that deviates from the baseline movement.

Optionally, analyzing comprises computing movement of the guidewire within the plurality of baseline images captured by an image sensor at a fixed pose, wherein the baseline movement of the guidewire comprises maximum ranges of movement of the guidewire during the at least one of heartbeats and breathing cycles, and wherein movement of the guidewire is detected when movement of the guidewire depicted in the successive images deviates from the baseline movement comprising the maximum ranges of movement.

Optionally, further comprising automatically detecting the fixed pose.

Optionally, analyzing comprises identifying at least one deformation of the guidewire within the plurality of baseline images captured by an image sensor at a fixed pose, wherein the baseline movement of the guidewire comprises the identified at least one deformation during the at least one of heartbeats and breathing cycles, and wherein movement of the guidewire is detected when deviation of the guidewire depicted in the successive images deviates from the at least one deformation of the baseline movement.

Optionally, analyzing comprises computing shape and/or movement of the guidewire relative to at least one fiducial marker that remains in a fixed location during the plurality of at least one of heart beats and breathing cycles, wherein the baseline movement of the guidewire is a range of shapes and/or movements relative to the at least one fiducial marker, wherein the movement of the guidewire that deviates from the baseline movement is determined by analyzing the shape and/or movement of the guidewire relative to the at least one fiducial marker.

Optionally, the at least one fiducial marker is selected from a group comprising: at least one anatomical structure of the subject, and an object affixed on the subject.

Optionally, analyzing comprises assigning a label to each of the plurality of baseline images indicating a phase during at least one of: a heartbeat cycle, and a breathing cycle, wherein monitoring comprises assigning the label to each successive image, and wherein detecting movement comprises determining a deviation of a location of the guidewire in each successive image of a certain label from the location of the guidewire in a baseline image with a label matching the certain label.

Optionally, analyzing comprises fitting a polynomial with a plurality of parameters to the guidewire depicted in the plurality of baseline images, wherein the plurality of parameters are adapted for fitting the polynomial to the guidewire depicted in each respective baseline image, wherein the baseline movement comprises at least one range of values of the plurality of parameters of the polynomial, wherein monitoring comprises computing values for the plurality of parameters for fitting the polynomial to the guidewire depicted in the successive images, and movement of the guidewire that deviates from the baseline movement is detected when the plurality of parameters are different than the at least one range.

Optionally, analyzing comprises fitting a spline comprising a plurality of piecewise curves to the guidewire depicted in the plurality of baseline images, wherein the piecewise curves are adapted for fitting the spline to the guidewire depicted in each respective baseline image, wherein the baseline movement comprises variations of each of the plurality of piecewise curves of the spline, wherein monitoring comprises fitting the spine to the guidewire depicted in the successive images, and movement of the guidewire that deviates from the baseline movement is detected by analyzing each of the plurality of piecewise curves of the spline to detect deviation of a certain piecewise curve from the baseline movement.

Optionally, further comprising creating at least one training record that includes at least one baseline image of the plurality of baseline images and a ground truth label indicating the baseline movement of the guidewire, training a machine learning model on the at least one training record, wherein monitoring comprises feeding the successive images into the machine learning model, and wherein detecting movement of the guidewire that deviates from the baseline moment is obtained as an outcome of the machine learning model.

Optionally, the at least one training record includes a sequence of the plurality of baseline images captured over a plurality of at least one of heartbeats and breathing cycles, wherein feeding comprises feeding a plurality of successive images captured over a plurality of at least one of heartbeats and breathing cycles.

Optionally, detecting movement comprises detecting at least one of: displacement of the guidewire in a certain direction, and at least one deformation of the guidewire, and further comprising generating an overlay for presentation on a display over at least one of the successive images indicating the at least one of: the certain direction of the displacement of the guidewire, and a portion of the guidewire undergoing the at least one deformation.

Optionally, further comprising analyzing the successive images to detect a change in pose of an image sensor that captures the images, and in response to the detected change, computing a new baseline movement of the guidewire from images captured after the change in pose is detected.

Optionally, further comprising analyzing the successive images to detect a change in pose of an image sensor that captures the images, and in response to the detected change, computing a transformation of a current position of the guidewire according to a transformation from a current pose of the image sensor to a preceding pose of the image sensor, and wherein the detecting movement of the guidewire that deviates from the baseline movement is according to the transformation of the current position of the guidewire.

Optionally, further comprising segmenting the guidewire from the plurality of images and of the successive images, wherein the analysis and the monitoring is according to the segmented guidewire.

Optionally, further comprising identifying tissues in proximity to the guidewire that are likely to be damaged by movement of the guidewire that deviates from the baseline movement, and wherein at least one of the analyzing, the computing the baseline movement, the monitoring, and the detecting movement, is for a portion of the guidewire in proximity to the identified tissues and is not performed for another portion of the guidewire that is not in proximity to the identified tissues.

Optionally, the body cavity comprises a left ventricle of the heart, wherein the guidewire is deformed and/or is moved within the left ventricle.

Optionally, the plurality of baseline images depict the guidewire at a target position, and wherein monitoring comprises monitoring the successive images of the guidewire for detecting movement of the guidewire from the target position that deviates from the baseline movement.

Optionally, the target position comprises an anchor position during a transcatheter medical procedure.

Optionally, the plurality of baseline images are a video of fluoroscopy images.

Optionally, further comprising generating an alert in response to detecting movement of the guidewire that deviates from the baseline movement.

Optionally, the alert is selected from a group comprising: a pop-up window presented on a display, an overlay over the guidewire indicating the detected movement, a sound, and a haptic feedback.

According to some embodiments of the present invention, there is provided a system for monitoring a guidewire during a medical procedure, comprising: at least one processor executing a code for: analyzing a plurality of baseline images captured over a plurality of at least one of heartbeats and breathing cycles, the plurality of baseline images, depicting a guidewire in a body cavity, computing according to the analysis, a baseline movement of the guidewire during the at least one of heartbeats and breathing cycles, and monitoring successive images of the guidewire for detecting movement of the guidewire that deviates from the baseline movement.

Optionally, analyzing comprises computing movement of the guidewire within the plurality of baseline images captured by an image sensor at a fixed pose, wherein the baseline movement of the guidewire comprises maximum ranges of movement of the guidewire during the at least one of heartbeats and breathing cycles, and wherein movement of the guidewire is detected when movement of the guidewire depicted in the successive images deviates from the baseline movement comprising the maximum ranges of movement.

Optionally, further comprising code for automatically detecting the fixed pose.

Optionally, analyzing comprises identifying at least one deformation of the guidewire within the plurality of baseline images captured by an image sensor at a fixed pose, wherein the baseline movement of the guidewire comprises the identified at least one deformation during the at least one of heartbeats and breathing cycles, and wherein movement of the guidewire is detected when deviation of the guidewire depicted in the successive images deviates from the at least one deformation of the baseline movement.

Optionally, analyzing comprises computing shape and/or movement of the guidewire relative to at least one fiducial marker that remains in a fixed location during the plurality of at least one of heart beats and breathing cycles, wherein the baseline movement of the guidewire is a range of shapes and/or movements relative to the at least one fiducial marker, wherein the movement of the guidewire that deviates from the baseline movement is determined by analyzing the shape and/or movement of the guidewire relative to the at least one fiducial marker.

Optionally, the at least one fiducial marker is selected from a group comprising: at least one anatomical structure of the subject, and an object affixed on the subject.

Optionally, analyzing comprises assigning a label to each of the plurality of baseline images indicating a phase during at least one of: a heartbeat cycle, and a breathing cycle, wherein monitoring comprises assigning the label to each successive image, and wherein detecting movement comprises determining a deviation of a location of the guidewire in each successive image of a certain label from the location of the guidewire in a baseline image with a label matching the certain label.

Optionally, analyzing comprises fitting a polynomial with a plurality of parameters to the guidewire depicted in the plurality of baseline images, wherein the plurality of parameters are adapted for fitting the polynomial to the guidewire depicted in each respective baseline image, wherein the baseline movement comprises at least one range of values of the plurality of parameters of the polynomial, wherein monitoring comprises computing values for the plurality of parameters for fitting the polynomial to the guidewire depicted in the successive images, and movement of the guidewire that deviates from the baseline movement is detected when the plurality of parameters are different than the at least one range.

Optionally, analyzing comprises fitting a spline comprising a plurality of piecewise curves to the guidewire depicted in the plurality of baseline images, wherein the piecewise curves are adapted for fitting the spline to the guidewire depicted in each respective baseline image, wherein the baseline movement comprises variations of each of the plurality of piecewise curves of the spline, wherein monitoring comprises fitting the spine to the guidewire depicted in the successive images, and movement of the guidewire that deviates from the baseline movement is detected by analyzing each of the plurality of piecewise curves of the spline to detect deviation of a certain piecewise curve from the baseline movement.

Optionally, further comprising code for creating at least one training record that includes at least one baseline image of the plurality of baseline images and a ground truth label indicating the baseline movement of the guidewire, training a machine learning model on the at least one training record, wherein monitoring comprises feeding the successive images into the machine learning model, and wherein detecting movement of the guidewire that deviates from the baseline moment is obtained as an outcome of the machine learning model.

Optionally, the at least one training record includes a sequence of the plurality of baseline images captured over a plurality of at least one of heartbeats and breathing cycles, wherein feeding comprises feeding a plurality of successive images captured over a plurality of at least one of heartbeats and breathing cycles.

Optionally, detecting movement comprises detecting at least one of: displacement of the guidewire in a certain direction, and at least one deformation of the guidewire, and further comprising generating an overlay for presentation on a display over at least one of the successive images indicating the at least one of: the certain direction of the displacement of the guidewire, and a portion of the guidewire undergoing the at least one deformation.

Optionally, further comprising code for analyzing the successive images to detect a change in pose of an image sensor that captures the images, and in response to the detected change, computing a new baseline movement of the guidewire from images captured after the change in pose is detected.

Optionally, further comprising code for analyzing the successive images to detect a change in pose of an image sensor that captures the images, and in response to the detected change, computing a transformation of a current position of the guidewire according to a transformation from a current pose of the image sensor to a preceding pose of the image sensor, and wherein the detecting movement of the guidewire that deviates from the baseline movement is according to the transformation of the current position of the guidewire.

Optionally, further comprising code for segmenting the guidewire from the plurality of images and of the successive images, wherein the analysis and the monitoring is according to the segmented guidewire.

Optionally, further comprising code for identifying tissues in proximity to the guidewire that are likely to be damaged by movement of the guidewire that deviates from the baseline movement, and wherein at least one of the analyzing, the computing the baseline movement, the monitoring, and the detecting movement, is for a portion of the guidewire in proximity to the identified tissues and is not performed for another portion of the guidewire that is not in proximity to the identified tissues.

Optionally, the body cavity comprises a left ventricle of the heart, wherein the guidewire is deformed and/or is moved within the left ventricle.

Optionally, the plurality of baseline images depict the guidewire at a target position, and wherein monitoring comprises monitoring the successive images of the guidewire for detecting movement of the guidewire from the target position that deviates from the baseline movement.

Optionally, the target position comprises an anchor position during a transcatheter medical procedure.

Optionally, the plurality of baseline images are a video of fluoroscopy images.

Optionally, further comprising code for generating an alert in response to detecting movement of the guidewire that deviates from the baseline movement.

Optionally, the alert is selected from a group comprising: a pop-up window presented on a display, an overlay over the guidewire indicating the detected movement, a sound, and a haptic feedback.

According to some embodiments of the present invention, there is provided a non-transitory medium storing program instructions for monitoring a guidewire during a medical procedure, which when executed by at least one processor, causes the at least one processor to: analyze a plurality of baseline images captured over a plurality of at least one of heartbeats and breathing cycles, the plurality of baseline images, depicting a guidewire in a body cavity, compute according to the analysis, a baseline movement of the guidewire during the at least one of heartbeats and breathing cycles, and monitor successive images of the guidewire for detecting movement of the guidewire that deviates from the baseline movement.

Optionally, analyzing comprises computing movement of the guidewire within the plurality of baseline images captured by an image sensor at a fixed pose, wherein the baseline movement of the guidewire comprises maximum ranges of movement of the guidewire during the at least one of heartbeats and breathing cycles, and wherein movement of the guidewire is detected when movement of the guidewire depicted in the successive images deviates from the baseline movement comprising the maximum ranges of movement.

Optionally, further comprising program instructions for automatically detecting the fixed pose.

Optionally, analyzing comprises identifying at least one deformation of the guidewire within the plurality of baseline images captured by an image sensor at a fixed pose, wherein the baseline movement of the guidewire comprises the identified at least one deformation during the at least one of heartbeats and breathing cycles, and wherein movement of the guidewire is detected when deviation of the guidewire depicted in the successive images deviates from the at least one deformation of the baseline movement.

Optionally, analyzing comprises computing shape and/or movement of the guidewire relative to at least one fiducial marker that remains in a fixed location during the plurality of at least one of heart beats and breathing cycles, wherein the baseline movement of the guidewire is a range of shapes and/or movements relative to the at least one fiducial marker, wherein the movement of the guidewire that deviates from the baseline movement is determined by analyzing the shape and/or movement of the guidewire relative to the at least one fiducial marker.

Optionally, the at least one fiducial marker is selected from a group comprising: at least one anatomical structure of the subject, and an object affixed on the subject.

Optionally, analyzing comprises assigning a label to each of the plurality of baseline images indicating a phase during at least one of: a heartbeat cycle, and a breathing cycle, wherein monitoring comprises assigning the label to each successive image, and wherein detecting movement comprises determining a deviation of a location of the guidewire in each successive image of a certain label from the location of the guidewire in a baseline image with a label matching the certain label.

Optionally, analyzing comprises fitting a polynomial with a plurality of parameters to the guidewire depicted in the plurality of baseline images, wherein the plurality of parameters are adapted for fitting the polynomial to the guidewire depicted in each respective baseline image, wherein the baseline movement comprises at least one range of values of the plurality of parameters of the polynomial, wherein monitoring comprises computing values for the plurality of parameters for fitting the polynomial to the guidewire depicted in the successive images, and movement of the guidewire that deviates from the baseline movement is detected when the plurality of parameters are different than the at least one range.

Optionally, analyzing comprises fitting a spline comprising a plurality of piecewise curves to the guidewire depicted in the plurality of baseline images, wherein the piecewise curves are adapted for fitting the spline to the guidewire depicted in each respective baseline image, wherein the baseline movement comprises variations of each of the plurality of piecewise curves of the spline, wherein monitoring comprises fitting the spine to the guidewire depicted in the successive images, and movement of the guidewire that deviates from the baseline movement is detected by analyzing each of the plurality of piecewise curves of the spline to detect deviation of a certain piecewise curve from the baseline movement.

Optionally, further comprising program instructions for creating at least one training record that includes at least one baseline image of the plurality of baseline images and a ground truth label indicating the baseline movement of the guidewire, training a machine learning model on the at least one training record, wherein monitoring comprises feeding the successive images into the machine learning model, and wherein detecting movement of the guidewire that deviates from the baseline moment is obtained as an outcome of the machine learning model.

Optionally, the at least one training record includes a sequence of the plurality of baseline images captured over a plurality of at least one of heartbeats and breathing cycles, wherein feeding comprises feeding a plurality of successive images captured over a plurality of at least one of heartbeats and breathing cycles.

Optionally, detecting movement comprises detecting at least one of: displacement of the guidewire in a certain direction, and at least one deformation of the guidewire, and further comprising generating an overlay for presentation on a display over at least one of the successive images indicating the at least one of: the certain direction of the displacement of the guidewire, and a portion of the guidewire undergoing the at least one deformation.

Optionally, further comprising program instructions for analyzing the successive images to detect a change in pose of an image sensor that captures the images, and in response to the detected change, computing a new baseline movement of the guidewire from images captured after the change in pose is detected.

Optionally, further comprising program instructions for analyzing the successive images to detect a change in pose of an image sensor that captures the images, and in response to the detected change, computing a transformation of a current position of the guidewire according to a transformation from a current pose of the image sensor to a preceding pose of the image sensor, and wherein the detecting movement of the guidewire that deviates from the baseline movement is according to the transformation of the current position of the guidewire.

Optionally, further comprising program instructions for segmenting the guidewire from the plurality of images and of the successive images, wherein the analysis and the monitoring is according to the segmented guidewire.

Optionally, further comprising program instructions for identifying tissues in proximity to the guidewire that are likely to be damaged by movement of the guidewire that deviates from the baseline movement, and wherein at least one of the analyzing, the computing the baseline movement, the monitoring, and the detecting movement, is for a portion of the guidewire in proximity to the identified tissues and is not performed for another portion of the guidewire that is not in proximity to the identified tissues.

Optionally, the body cavity comprises a left ventricle of the heart, wherein the guidewire is deformed and/or is moved within the left ventricle.

Optionally, the plurality of baseline images depict the guidewire at a target position, and wherein monitoring comprises monitoring the successive images of the guidewire for detecting movement of the guidewire from the target position that deviates from the baseline movement.

Optionally, the target position comprises an anchor position during a transcatheter medical procedure.

Optionally, the plurality of baseline images are a video of fluoroscopy images.

Optionally, further comprising program instructions for generating an alert in response to detecting movement of the guidewire that deviates from the baseline movement.

Optionally, the alert is selected from a group comprising: a pop-up window presented on a display, an overlay over the guidewire indicating the detected movement, a sound, and a haptic feedback.

According to some embodiments of the present invention, there is provided a computer implemented method of monitoring a guidewire position during a medical procedure, comprising: receiving a target position of the guidewire, analyzing images of the guidewire, captured over a plurality of at least one of heartbeats and breathing cycles, for detecting a current position of the guidewire, and alerting when the current position of the guidewire deviates from the target position.

Optionally, further comprising: computing the target position from an anatomical image obtained prior to the medical procedure, and correlating the target position of the anatomical image to the images captured over the plurality of at least one of heartbeats and breathing cycles, wherein determining when the current position deviates from the target position is according to the correlation.

Optionally, the target position is a boundary region computed for an anatomical image, and further comprising computing a transformation matrix according to the correlating, and transforming the boundary region computed for the anatomical image to the images using the transformation matrix.

Optionally, further comprising segmenting the guidewire, and determining when the current position deviates from the target position by determining whether the segmented guidewire is within the transformed boundary region.

Optionally, the target position of the guidewire is obtained from an anatomical image that excludes presence of the guidewire.

Optionally, the target position is defined by a boundary including bounding box and/or non-uniform region of an anatomical image that delineates a boundary defining the target location for the guidewire.

Optionally, the target position is automatically defined by a machine learning model that receives the anatomical image as input and outputs the boundary, wherein the machine learning model is trained on a training dataset of multiple images depicting the body cavity within which the guidewire will be placed, with ground truth marking of boundaries.

Optionally, the target position is represented as a boundary, and further comprising: applying the boundary to an initial successive image at a certain phase of the plurality heartbeat and/or breathing cycles, computing a transformation from a current successive image to a subsequent successive image at another phase of the heartbeat and/or breathing cycle, applying the transformation to the boundary representing the target position of the subsequent successive image, and determining whether the guidewire is within the transformed boundary or not.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### In the Drawings:

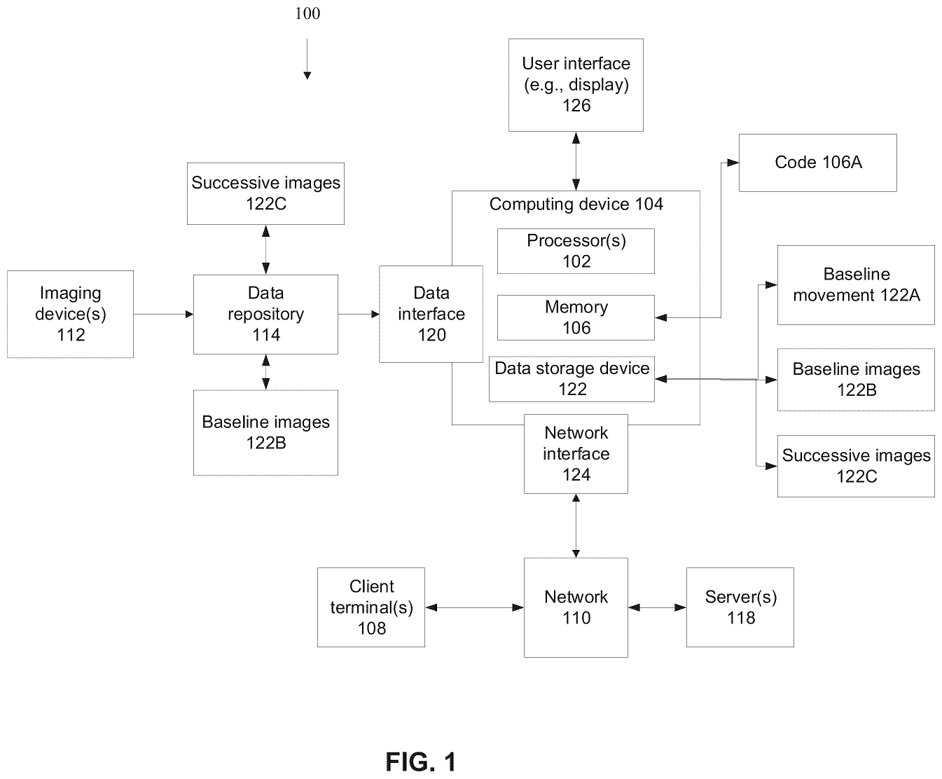

FIG. 1 is a block diagram of components of a system 100 for monitoring a guidewire position during a medical procedure, in accordance with some embodiments of the present invention;

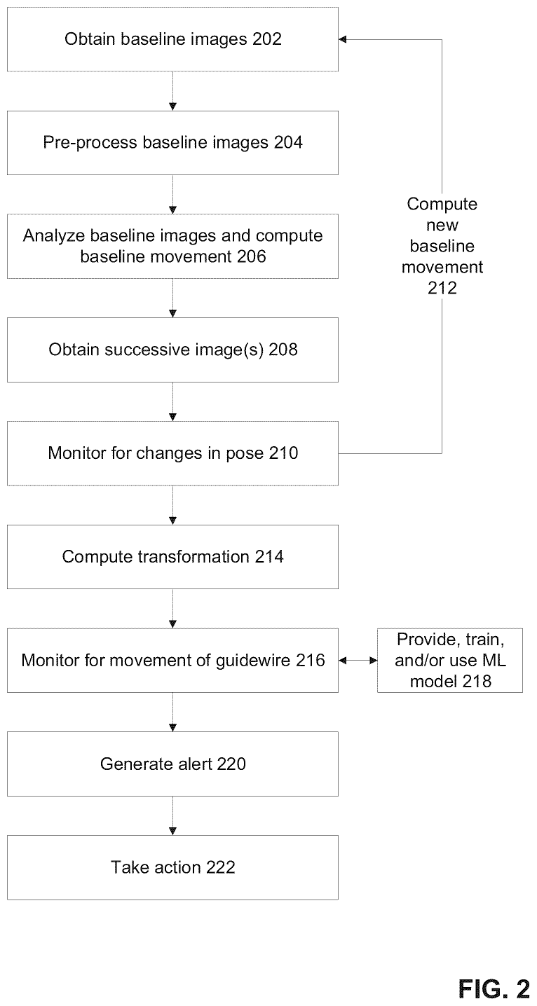

FIG. 2 is a flowchart of a method of monitoring a guidewire position during a medical procedure, in accordance with some embodiments of the present invention;

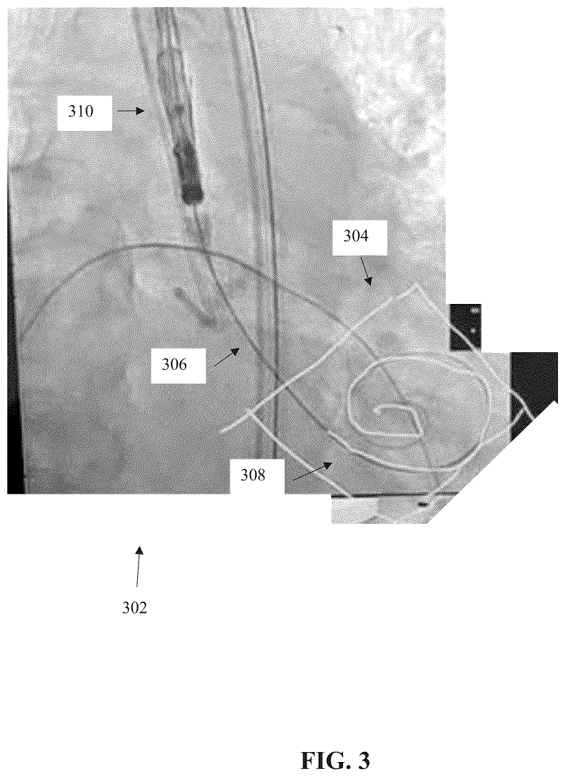

FIG. 3 is a schematic of an image captured during a medical procedure, overlaid with a boundary box denoting a baseline movement of a portion of a guidewire, in accordance with some embodiments of the present invention; and

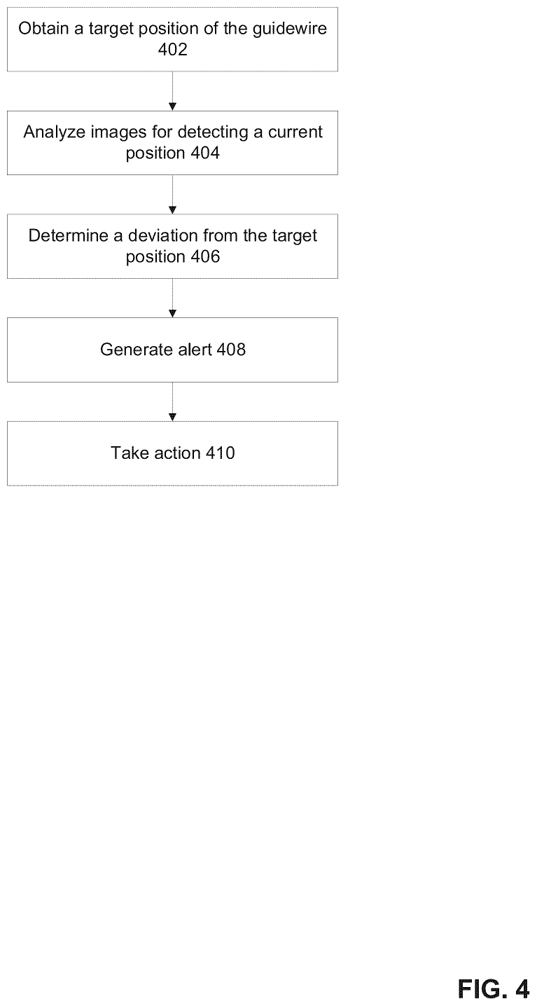

FIG. 4 is a flowchart of another exemplary method of monitoring a guidewire position during a medical procedure, in accordance with some embodiments of the present invention.

### DESCRIPTION OF SPECIFIC EMBODIMENTS OF THE INVENTION

The present invention, in some embodiments thereof, relates to image processing and, more specifically, but not exclusively, to systems and methods for analyzing medical images.

An aspect of some embodiments of the present invention relates to systems, methods, computing devices, and/or code instructions (stored on a data storage device and executable by one or more processors) for monitoring a guidewire position during a medical procedure, for example, a transcatheter procedure being performed in the heart, such as a valve replacement (e.g., transcatheter aortic valve implantation (TAVI)), atrial fibrillation ablation, and closure of an atrial septal defect (ASD) and/or patent foramen ovale (PFO). In some embodiments, the guidewire is a SAFARI guidewire by Boston Scientific or SavvyWire™ by OpSens Medical, used in TAVI procedures.

These procedures commonly involve implantations and/or physical manipulations of heart tissue, often via transcatheter delivery. There is an incidence of new onset conductance disturbances (NOCDs) associated with certain treatment; for example, in procedures and/or implantations to correct: atrial and ventricular septal defects (e.g., via percutaneous device-based closures), aortic stenosis (e.g., via transcatheter aortic valve replacement, TAVR), and tricuspid regurgitation (e.g., via transcatheter implantation of devices for annuloplasty, coaptation, and/or valve replacement).

A processor may analyze multiple baseline images (e.g., 2D fluoroscopy images) captured over multiple heartbeats and/or breathing cycles. The baseline images depict the guidewire in a body cavity, optionally at a target position. The target position of the guidewire may refer to an anatomical location of the body where a certain portion of the guidewire is located, for example, a pigtail portion of the guidewire is located in the left ventricle (e.g., just prior to the treatment procedure), about 2-3 centimeters below the aortic valve. A baseline movement of the guidewire during the heartbeats and/or breathing cycles may be computed according to the analysis. The baseline movement may represent the range of motion of the guidewire located in the body cavity resulting from the heartbeats and/or breathing cycles, for example, the limits of motion of the guidewire due to cyclical heartbeats and/or breathing. The baseline movement may be along a cyclical path, in one or more degrees of freedom, for example, up/down, left/right, along a line, along a circle, and/or along another curve. For example, each beat of the left ventricle deforms the guidewire located in the left ventricle. In another example, each cycle of inhalation and expiration raises and lowers the chest of the subject, which appears as an up-down movement of the guidewire on the images. Successive images of the guidewire may be monitored for detecting movement of the guidewire that deviates from the baseline movement, optionally from the target position. For example, movement outside of the range of motion resulting from the heartbeats and/or breathing cycles. Such movement outside of the range of motion defined by the baseline movement may represent undesired movement of the guidewire, that does not result from the heartbeats and/or breathing, but may result, for example, from an erroneous pull on the guidewire from outside of the body of the subject. The erroneous pull may cause a slight deviation from the baseline movement while the guidewire remains within the target position, or may be a larger deviation from the baseline movement where the guidewire is removed from the target position. An operator (e.g., physician) may prevent further undesired motion of the guidewire, and/or restore the guidewire to its previous target position. Movement of the guidewire from the baseline movement, optionally from the target position, may result in occurrence of conductance disturbances.

An aspect of some embodiments of the present invention relates to systems, methods, computing devices, and/or code instructions (stored on a data storage device and executable by one or more processors) for another approach of monitoring a guidewire position during a medical procedure. A processor may receive a target position of the guidewire, for example, obtained from a still pre-procedure image that excludes the guidewire and is captured at a random phase of the heartbeat cycle and/or breathing cycle, such as a CT scan. The processor may analyze images of the guidewire captured during the procedure. The image are captured over multiple heartbeats and/or breathing cycles, for detecting a current position of the guidewire. The processor may determine when the current position of the guidewire deviates from the target position, optionally for generating an alert. For example, when the guidewire, which is supposed to remain within the left ventricle during a certain time interval of the procedure, is moved out of the left ventricle.

At least some embodiments described herein address the technical problem and/or medical problem of monitoring for motion of a guidewire that is located inside a body cavity of a subject (e.g., left ventricle (LV) of a heart, a right ventricle, a left atrium, a right atrium, an aorta, a right ventricular outflow tract, bladder, renal artery, kidney, carotid artery, blood vessels of the brain, and the like), for example, for helping to assure that the guidewire does not change its intrabody position from a target position such as during a certain time interval of a procedure. During a medical procedure (e.g., transcatheter aortic valve implantation (TAVI), PFO and/or ASD closure, ablation for treatment of atrial fibrillation), a device delivery system may be introduced over a guidewire. The guidewire may be positioned in the body cavity (e.g., LV) and may function as an anchor during the procedure. When the delivery system and/or treatment device is inserted and/or taken out, optionally threaded over the guidewire, the guidewire may be moved from its anchor position (e.g., due to friction or erroneous pulling or pushing of the guidewire). The inadvertent movement of the guidewire may cause damage in the conduction system of the heart and/or may cause damage to nearby tissues (and/or other organs when the cavity is in another organ), for example, by shear forces applied to the tissues lining the interior of the heart.

At least some embodiments described herein improve the technical field of image processing, and/or the medical field of increasing patient safety during a transcatheter procedure.

In at least some embodiments, the improvement is in the ability to detect movement of the guidewire within images captured during heartbeats and/or breathing cycles, where the motion of the heart beating and/or motion of the lungs and/or diaphragm for breathing causes movement of the guidewire even when the guidewire is anchored in a fixed target location. For example, the beating heart deforms the shape of the guidewire, and/or the guidewire moves up and down with the chest during breathing. Movement of the guidewire may be determined as deviating from the natural body movement that occurs from the heartbeats and/or breathing cycles, referred to herein as baseline movement. The deviation of movement of the guidewire from the baseline movement may be determined during one or more phases of the procedures such as delivery of the catheter and/or treatment device (e.g., introduction, extraction, positioning, and/or deployment of a device (e.g., transcatheter aortic valve replacement (TAVR)).

Some embodiments may identify when the guidewire changed its position and/or has been deformed and may generate alerts indicating the detected movement that deviates from the baseline movement. The alert may allow the operator to take action for avoiding further undesired movement of the guidewire, which may reduce or prevent the damage to the interior walls of the tissues and/or organ, for example, to the conduction system of the heart.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference is now made to FIG. 1, which is a block diagram of components of a system 100 for monitoring a guidewire position during a medical procedure, in accordance with some embodiments of the present invention. Reference is also made to FIG. 2, which is a flowchart of a method of monitoring a guidewire position during a medical procedure, in accordance with some embodiments of the present invention. Reference is also made to FIG. 3, which is a schematic of an image 302 captured during a medical procedure, overlaid with a boundary box 304 denoting a baseline movement of a portion of a guidewire 306, in accordance with some embodiments of the present invention. Reference is also made to FIG. 4, which is a flowchart of another exemplary method of monitoring a guidewire position during a medical procedure, in accordance with some embodiments of the present invention.

System 100 may implement the acts of the method described with reference to FIGS. 2 and/or 4, optionally by one or more hardware processor(s) 102 of a computing device 104 executing code instructions stored in a memory 106.

Computing device 104 may be implemented as, for example, a client terminal, a server, a virtual server, a radiology workstation, a catheterization laboratory workstation, a virtual machine, a computing cloud, a mobile device, a desktop computer, a thin client, a Smartphone, a Tablet computer, a laptop computer, a wearable computer, glasses computer, and a watch computer. Computing device 104 may include an advanced visualization process that sometimes is add-on to a catheterization laboratory workstation and/or other devices for presenting overlays on fluoroscopy images, e.g., for guiding a trans-catheter medical procedure (e.g., aortic valve replacement intervention, PTO and/or ASD closure, ablation) in a subject.

Computing device 104 may include locally stored software that performs one or more of the acts described with reference to FIGS. 2 and/or 4, and/or may act as one or more servers (e.g., network server, web server, a computing cloud, virtual server) that provides services (e.g., one or more of the acts described with reference to FIGS. 2 and/or 4) to one or more client terminals 108 (e.g., remotely located catheterization laboratory workstation) over a network 110, for example, providing software as a service (SaaS) to the client terminal(s) 108, providing an application for local download to the client terminal(s) 108, as an add-on to a web browser and/or a medical imaging viewer application, and/or providing functions using a remote access session to the client terminals 108, such as through a web browser.

Different architectures based on system 100 may be implemented. In one example, computing device 104 provides centralized services. Computing device 104 may obtain baseline medical images 122B (e.g., fluoroscopy images) and/or successive images 122C (which are obtained after the baseline movement 122A has been computed for monitoring for motion of the guidewire deviating from the baseline movement) from each of multiple different imaging devices 112 (e.g., fluoroscopy machines). Each imaging device 112 includes an image sensor that captures the images. The baseline images 122B and/or successive images 122C may be provided to computing device 104 for centralized computing of baseline movement 122A, and/or for centralized evaluation to determine deviation from the baseline movement 122A. Baseline images 122B and/or successive images 122C may be provided to computing device 104, for example, via an application programming interface (API), a local application, over a network 110, and/or transmitted using a suitable transmission protocol. The indication of whether a deviation from the baseline movement 122A has been determined, and/or what the deviation is (e.g., forward displacement, reverse displacement, deviation in shape) may be provided, for example, to client terminal(s) 108 for presentation on a display and/or other indications (e.g., audio and the like) and/or local storage, stored in an electronic medical record (e.g., hosted by server 118), and/or stored by computing device 104.

In another architecture, computing device 104 provides localized services, by locally analyzing the baseline images 122B for computing the baseline movement 122A and/or determining deviation from the baseline movement 122A from successive images 122C. For example, computing device 104 is implemented as code executed by a processor of a catheterization laboratory workstation that analyses fluoroscopy images captured by a fluoroscopy machine of the catheterization laboratory.

Imaging device 112 provides the medical images. Optionally, imaging device 112 is a 2D fluoroscopy and/or x-ray machine, as commonly used in catheterization labs (e.g., cardiac). A subset of the images may be designated as baseline images 122B, as described herein.

Baseline images 122B and/or successive images 122C may be stored in a data repository 114 and/or data storage device 122, for example, a storage server, a computing cloud, virtual memory, and a hard disk. Baseline images 122B may be analyzed for determining baseline movement 122A, as described herein. Successive images 122C may be monitored for determining a deviation from baseline movement 122A, as described herein. It is noted that baseline images 122B and/or successive images 122C may be stored by a server 118, accessibly by computing device 104 over network 110.

Computing device 104 may receive baseline images 122B and/or successive images 122C for analysis for deviation from the baseline movement from imaging device 112 and/or data repository 114 using one or more data interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)), and/or a network interface 124.

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) may store code instruction for execution by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). For example, memory 106 may store code 106A that implement one or more acts and/or features of the method described with reference to FIGS. 2 and/or 4.

Computing device 104 may include a data storage device 122 for storing data, for example, an indication of the computed baseline movement 122A (e.g., as described herein) and/or baseline images 122B and/or successive images 122C, as described herein. Data storage device 122 may be implemented as, for example, a memory, a local hard-drive, a removable storage device, an optical disk, a storage device, and/or as a remote server and/or computing cloud (e.g., accessed over network 110).

Computing device 104 may include network interface 124 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 104 may access one or more remote servers 118 using network 110.

It is noted that data interface 120 and network interface 124 may exist as two independent interfaces (e.g., two network ports), as two virtual interfaces on a common physical interface (e.g., virtual networks on a common network port), and/or integrated into a single interface (e.g., network interface). Computing device 104 may communicate using network 110 (or another communication channel, such as through a direct link (e.g., cable, wireless) and/or indirect link (e.g., via an intermediary computing device such as a server, and/or via a storage device) with one or more of:
* Client terminal(s) 108, for example, when computing device 104 acts as a server providing image analysis services (e.g., SaaS) to remote catheterization laboratory workstations, for analyzing remotely obtained baseline images 122B (e.g., fluoroscopy images) for computing the baseline movement 122A and/or computing deviation from baseline movement 122A from successive images 122C.
* Server 118, for example, implemented in association with a picture arching and communication system (PACS), which may store captured baseline images 122B used to compute baseline movement 122A and/or which may store successive images 122C to determine deviation from baseline movement 122A.
* Imaging device 112 and/or data repository 114 that store images acquired by imaging device 112. The acquired fluoroscopy images may be baseline images 122B used to compute baseline movement 122A and/or successive images 122C that are analyzed to determine a deviation from baseline movement 122A.

Computing device 104 and/or client terminal(s) 108 and/or server(s) 118 may include and/or may be in communication with a user interface(s) 126 that includes a mechanism designed for a user to enter data (e.g., indicate start of the monitoring for movement of the guidewire and/or end of the monitoring) and/or to view data (e.g., indication of deviation of movement of the guidewire from the baseline movement) such as an overlay over the input image. Exemplary user interfaces 126 include, for example, one or more of, a touchscreen, a display, a keyboard, a mouse, and voice activated software using speakers and microphone.

Referring now back to FIG. 2, at 202, the processor(s) may obtain multiple baseline images, for example, images depicting a guidewire in a body cavity. The guidewire position may be, for example, in the left ventricle during a TAVI procedure.

As used herein, the term processor performing a task refers to the processor executing code for performing the task. The term processor may refer to a computing device and/or system (e.g., as described with reference to FIG. 1) that includes the processor.

The body cavity may be, for example, a left ventricle of the heart, a right ventricle, a left atrium, a right atrium, an aorta, a right ventricular outflow tract, bladder, renal artery, kidney, carotid artery, blood vessels of the brain, and the like.

The images may be a video of fluoroscopy images (e.g., CINE), and/or a sequence of individual fluoroscopy images. The images may be two dimensional (2D).

When the guidewire is located at a target position, the images are referred to herein as baseline images. In some embodiments, the user (e.g., the physician) may indicate, e.g., via user interface 126, when the guidewire is at a target position.

The target position may refer to the location of the guidewire within the body during a certain phase of a procedure where movement of the guidewire from the target position is undesired, for example, due to risk of damage to the conduction system from movement of the wire. The target position may be an anchor position during a transcatheter medical procedure. The target position may represent a position of the guidewire prior to starting the transcatheter medical procedure, where the position of the guidewire is to be maintained unless explicitly moved on purpose by an operator performing the transcatheter medical procedure.

The baseline images may be obtained over a time interval, for example, at least about 5 seconds, or 10 seconds, or 15 seconds, or greater. The time interval may be selected to cover at least one full harmonic cycle which includes the frequency of the heartbeats and the frequency of the breathing cycle. Since the heart beats at a frequency that is different than the frequency of breathing, the time interval may be selected to include base images that depict sufficient multiple different combinations of the state of the beating heart and the state of breathing.

The baseline images may be captured over multiple heartbeats and/or breathing cycles, during which the guidewire remains stationary at the target position within body cavity, apart from natural motion due to the heartbeats and/or breathing. The baseline images depict the motion and/or deformation of the guidewire from the heartbeats and/or breathing cycles. For example, when the guidewire is in the left ventricle, the contraction and relaxation of the left ventricle deform the shape of the guidewire. In another example, when the guidewire is in the left ventricle, the inhalation and expiration of the subject move the chest up and down, resulting in motion of the guidewire.

As used herein, motion of the guidewire may include deformation of the guidewire and/or changes of shape of the guidewire. The terms deformation and changes of shape may sometimes be used interchangeably.

Multiple baseline images captured over multiple heartbeats and/or breathing cycles may be expected to depict a full range of cyclical motion and/or a full range of cyclical deformation, which may be predicted to repeatedly occur during future heartbeats and/or breathing cycles.

At 204, the processor(s) may pre-process the baseline images.

Optionally, the baseline images are pre-processed by automatically detecting the fixed pose of the image sensor of the imaging device. The fixed pose may be, for example, the orientation of the arm of the imaging device that includes the image sensor. For example, the c-arm of a fluoroscopy machine is fixed in a certain position and/or orientation to define the fixed pose. The method may proceed (e.g., to 206 and/or other features for computing movement of the guidewire in response to the detected fixed pose) when the fixed pose is detected.

The fixed pose of the image sensor may be detected, for example, by detecting no change in the baseline image over a time interval, for example, at least 10 seconds, or 30 seconds, or 1 minute, or 2 minutes, or other values. The lack of change in the baseline images may be detected, for example, by subtracting images from each other over time. When the pose is fixed, the result of the subtraction should be zero or near zero (e.g., due to artifacts). In another example, the fixed pose may be detected by identifying features on the image (e.g., edge detection process, feature extraction, neural network), and tracking the features across images. When the features move and/or disappear, the pose is assumed to have changed. In yet another example, the fixed pose may be detected by a motion sensor, accelerometer, angular sensor, and/or location sensor (or other sensors), placed on the image sensor.

Alternatively or additionally, the baseline images are pre-processed by segmenting the guidewire from the baseline images. Further features of the method (e.g., analysis, monitoring, and/or others) may be computed according to the segmented guidewire. The guidewire may be segmented in other features, for example, images that are monitored for movement of the guidewire. The guidewire may be segmented, for example, by a machine learning model (ML) trained on images with ground truth segmentations of the guidewire, identifying edges and/or curves indicating the guidewire, and identifying pixels with intensity values indicating the guidewire.

Alternatively or additionally, the baseline images are pre-processed by identifying tissues in proximity to the guidewire that are likely to be damaged by movement of the guidewire from the baseline movement and/or target position. The features of the method (e.g., analyzing, computing the baseline movement, monitoring, and detecting movement) may be performed for a portion of the guidewire in proximity to the identified tissues and are not performed for another portion of the guidewire that is not in proximity to the identified tissues. The tissues may be identified, for example, by a machine learning model (ML) trained on images with ground truth labels of the tissue, identifying edges and/or curves indicating the guidewire and marking a bounding box expected to include the tissues, and identifying pixels with intensity values indicating the guidewire and marking the bounding box.

At 206, the processor(s) may analyze the baseline images captured over multiple heartbeats and/or breathing cycles. The processor may compute a baseline movement of the guidewire during the heartbeats and/or breathing cycles according to the analysis. The baseline movement may represent external boundaries in two dimensions such as x-y (which can be overlaid over successive images), indicating the expected movement of the guidewire arising from the heartbeats and/or breathing cycles. The external boundaries may be, for example, a bounding box, and/or non-uniform shape traced over an area as the guidewire appears to “move” in successive frames of the baseline images.

The analysis and computation of the baseline movement may be triggered in response to detection of the fixed pose of the image sensor, which may be automatically detected as described with reference to 204 of FIG. 2.

When the images are captured by a 2D image sensor, the fixed pose may help ensure accuracy of the analysis of the 2D images. A change in pose may appear in successive 2D images as a change in movement of the guidewire, when in fact the guidewire has remained at the baseline movement.

Optionally, the analysis is performed by computing movement of the guidewire within the baseline images. The baseline movement of the guidewire may be defined as the maximum ranges of movement of the guidewire during the heartbeats and breathing cycles. The baseline movement may be computed by subtracting successive images from each other to obtain pixels indicating the guidewire (which may be radio-opaque and have pixel intensity different from surrounding tissue). A filter may be applied to remove artifacts and/or features of tissues resulting from movement of the tissues due to the heartbeats and/or breathing cycles. The boundary movement is computed by aggregating the pixels of guidewires from the multiple subtractions, for example, the boundary movement is an overlay of the image that includes pixels (e.g., x,y coordinates) that depicted the guidewire in multiple baseline images over multiple heartbeats and/or breathing cycles. Movement of the guidewire (e.g., as described with reference to 214) may be detected when movement of the guidewire depicted in successive monitored images deviates from the baseline movement, i.e., is larger than the maximum ranges of movement, and/or when the guidewire appears outside the computed overlay region.

Alternatively or additionally, the analysis is performed by identifying deformation(s) of the guidewire within the baseline images, for example, changes in shape of the guidewire. The baseline movement of the guidewire may be defined as the identified deformation(s) during the heartbeats and/or breathing cycles. Movement of the guidewire (e.g., as described with reference to 214) may be detected when deviation of the guidewire depicted in the successive monitored images deviates from the deformation(s) of the baseline movement.

Alternatively or additionally, the analysis is performed by computing shape and/or movement of the guidewire relative to one or more fiducial markers that remain in a fixed location during the heartbeats and breathing cycles. The fiducial marker(s) may be anatomical structure(s) of the subject that remain fixed during heartbeats and/or breathing cycles, for example, vertebrae. In another example, the fiducial marker(s) may be objects (e.g., radio-opaque) that are affixed on the subject and/or placed in proximity to the subject. The baseline movement of the guidewire may be defined as a range of shape and/or movements relative to the fiducial marker(s). Movement of the guidewire that deviates from the baseline movement may be determined by analyzing the shape and/or movement of the guidewire relative to the fiducial marker(s).

Alternatively or additionally, the analysis is performed by mapping the baseline images (e.g., each image, and/or a subset of the images) to a phase of the heartbeat and/or breathing cycle. The mapping may be done, for example, by assigning a label to each baseline image being mapped, and/or correlating the baseline image with an indication of the phase. For example, an ECG of the subject is simultaneously obtained with the baseline images. The time of each baseline image is mapped to the time of the ECG signal, which may be automatically analyzed to determine the phase of the cardiac cycle, for example, P wave, PR segment, QRS complex, ST segment, T wave, U wave, or others. In another example, the time of each baseline image is mapped to output of an anesthesia machine that is providing artificial respiration to the subject, and/or to a ventilation machine that is monitoring breathing of the subject. In another example, the image itself is analyzed to determine the phase, for example, by training a machine learning model to recognize the phase, such as by training the machine learning model on a training dataset of multiple images each labelled with a ground truth label indicating the phase. In such embodiments, each phase of the heartbeat and/or breaching cycle is associated with a different baseline movement, indicating the baseline location of the guidewire at that phase. Movement of the guidewire that deviates from the baseline movement may be determined by mapping each (or some) successive image to a certain phase (e.g., assigning the label of the phase to the image), and determining a deviation of a location of the guidewire from the baseline movement of the phase. For example, the label of the successive image is used to find the matching label of the baseline image defining the baseline location. The successive image may be compared to the baseline image of the same phase, for example, by subtracting the two images, computing a correlation between the two images, computing optical flow between the two images to determine what features moved between the two images, and feeding the two images into a machine learning model trained to find the differences.

Alternatively or additionally, the analysis is performed by fitting a polynomial with parameters to the guidewire depicted in the baseline images. The parameters are computed for fitting the polynomial to the guidewire depicted in respective baseline images (e.g., each image, some images). The baseline movement may be defined as range(s) of values of the parameters of the polynomial computed for the guidewire for the baseline images during the multiple heartbeats and/or breathing cycles. The monitoring may be performed by computing values for the parameters for fitting the polynomial to the guidewire depicted in respective successive images. Movement of the guidewire from the baseline movement and/or target position may be detected when the parameters are different than the range(s).

Alternatively or additionally, the analysis is performed by fitting a spline to the guidewire depicted in the baseline images. The spline includes piecewise curves that are compute for fitting the spline to the guidewire depicted in respective baseline images (e.g., each image, some images). The baseline movement may be defined as variations of each of the piecewise curves of the spline during the multiple heartbeats and/or breathing cycles, for example, the range over a 2D area covered by the multiple instances of each piecewise curve over the baseline images. The monitoring may be performed by fitting the spine to the guidewire depicted in respective successive images. Movement of the guidewire from the baseline movement and/or target position may be detected by analyzing each of the piecewise curves of the spline to detect deviation of a certain piecewise curve from the baseline movement.

Alternatively or additionally, the baseline movement, or portion thereof, may be pre-defined, manually by a user and/or automatically by the processor (e.g., pre-learned, dynamically learned, and the like). The baseline movement may include regions into which movement is prohibited, for example, internal walls of tissues which may be automatically detected by the processor, a border within a blood vessel and/or cavity past which the guidewire should not be displaced (e.g., do not push guidewire into the pulmonary artery), and/or a present tolerance (e.g., no more than about 5%, 10%, 15%, 20% or other values, greater than the range of motion of the baseline movement).

At 208, the processor obtains successive images. The successive images are obtained after the baseline images have been obtained. The successive images are assumed to be obtained at the fixed pose, which was used for computing the baseline movement. The successive images are of the same type as the baseline images, optionally fluoroscopy images, such as 2D fluoroscopy images.

At 210, the processor analyzes the successive images to detect a change in pose of the image sensor. The change in pose may be detected, for example, by subtracting images from each other over time. When the pose has changed, the result of the subtraction is not zero or near zero. In another example, the change in pose may be detected by identifying features on the image (e.g., edge detection process, feature extraction, neural network), and detecting a statistically significant movement of the features across images. When the features move and/or disappear, the pose is assumed to have changed. In yet another example, the change in pose may be detected by a motion sensor, accelerometer, angular sensor, and/or location sensor (or other sensors), placed on the image sensor. In yet another example, the change in pose may be detected by a trained ML model, that is trained on a training dataset of images labelled with a certain pose. When the pose of the image sensor changes, the ML model outputs an indication that the pose is different. The ML model may be dynamically trained during the procedure, for example, by fixing the pose for a certain time (e.g., 1 minute, 3 minutes, or other values) and automatically labelling the images with a ground truth label of the pose (e.g., LAO, RAO view, or others). The pose may be detected using optical character recognition (OCR) applied to the image when the image pose (e.g., LAO, RAO, etc.) is indicated in the fluoroscopy image.

Optionally, when the baseline movement of the guidewire is defined as the maximum ranges of movement of the guidewire during the heartbeats and breathing cycles, movement of the guidewire may be detected when the guidewire depicted in successive monitored images deviates from the baseline movement, i.e., is larger than the maximum ranges of movement.

Alternatively or additionally, when the baseline movement is defined as the overlay region overlaid on the successive images, movement of the guidewire may be detected when the guidewire in successive monitored images deviates outside of the overlay region.

Alternatively or additionally, when the baseline movement of the guidewire is defined as the identified deformation(s) during the heartbeats and/or breathing cycles, movement of the guidewire may be detected when deviation of the guidewire depicted in the successive monitored images deviates (e.g., is statistically different, and/or is larger and/or is smaller) from the deformation(s) of the baseline movement.

Alternatively or additionally, when the baseline movement of the guidewire is defined as a range of shape and/or movements relative to fiducial marker(s), movement of the guidewire that deviates from the baseline movement may be determined by analyzing the shape and/or movement of the guidewire relative to the fiducial marker(s).

Alternatively or additionally, when each phase of the heartbeat and/or breaching cycle is associated with a different baseline movement, indicating the baseline location of the guidewire at that phase, movement of the guidewire that deviates from the baseline movement may be determined by mapping each (or some) successive image to a certain phase (e.g., assigning the label of the phase to the image), and determining a deviation of a location of the guidewire from the baseline movement of the phase. For example, the label of the successive image is used to find the matching label of the baseline image defining the baseline location. The successive image may be compared to the baseline image of the same phase, for example, by subtracting the two images, computing a correlation between the two images, computing optical flow between the two images to determine what features moved between the two images, and feeding the two images into a machine learning model trained to find the differences.

Alternatively or additionally, when baseline movement is defined as range(s) of values of the parameters of the polynomial computed for the guidewire for the baseline images during the multiple heartbeats and/or breathing cycles, the monitoring may be performed by computing values for the parameters for fitting the polynomial to the guidewire depicted in respective successive images. Movement of the guidewire from the baseline movement and/or target position may be detected when the parameters are different than the range(s).

Alternatively or additionally, when the baseline movement is defined as variations of each of the piecewise curves of the spline during the multiple heartbeats and/or breathing cycles, the monitoring is performed by fitting the spine to the guidewire depicted in respective successive images. Movement of the guidewire from the baseline movement and/or target position may be detected by analyzing each of the piecewise curves of the spline to detect deviation of a certain piecewise curve from the baseline movement.

At 212, in response to detecting the change in pose of the image sensor, features described with reference to 202-210 are iterated, for computing a new baseline movement of the guidewire from images captured after the change in pose is detected. The new baseline movement is determined for the new pose.

Alternatively to 212, at 214, in response to detecting the change in pose, the processor may compute a transformation of a current position of the guidewire, according to the transformation from a current pose of the image sensor to a preceding pose of the image sensor. The movement of the guidewire that deviates from the baseline movement and/or target position may be detected according to the transformation of the current position of the guidewire.

At 216, the processor may monitor the successive images of the guidewire for detecting movement of the guidewire that deviates from the baseline movement and/or from the target position.

At 218, a machine learning model may be provided, trained, and/or used (i.e., inference) for detecting movement of the guidewire.

The ML model may be trained by creating a training dataset of multiple records. Each record may include one or more baseline images and a ground truth label indicating the baseline movement of the guidewire. Optionally, one or more training records include a sequence of baseline images captured over one or more phases of one or more heartbeats and/or breathing cycles. There may be multiple records of multiple baseline images and corresponding ground truth labels to cover the multiple phases of the heartbeat cycle and/or the breathing cycle.

The training dataset may be a customized database that is dynamically created during the procedure. For example, baseline images collected over a time interval (e.g., 15 seconds, 1 minute, 3 minutes, or other) are automatically labelled as baseline movement by the processor. The operator may indicate start and stop of the time interval, for example, by pressing an icon on a user interface. The training dataset may be re-generated each time the pose of the image sensor has changed, for example, by the operator pressing an icon on the user interface to trigger the processor labelling new baseline images.

The ML model may be trained on the training dataset. The monitoring of the successive images may be performed by feeding the successive images into the machine learning model. In some implementations, according to the structure of the training records, individual successive images are fed into the ML model. In other implementations, according to the structure of the training records, a sequence of successive images captured over one or more phases of one or more heartbeats and/or breathing cycles is fed into the ML model. An indication of the detected movement of the guidewire that deviates from the baseline moment is obtained as an outcome of the machine learning model. For example, the ML model may output a binary value indicative whether movement of the guidewire relative to the baseline movement was detected or not. In another example, the ML model may output a value indicating magnitude of the deviation of the guidewire from the baseline movement.

Exemplary architectures of ML models described herein include, for example, one or more of: a detector architecture, a classifier architecture, neural networks of various architectures (e.g., convolutional, fully connected, deep, encoder-decoder, recurrent, graph), support vector machines (SVM), logistic regression, k-nearest neighbor, decision trees, boosting, random forest, a regressor, and/or any other commercial or open source package allowing regression, classification, dimensional reduction, supervised, unsupervised, semi-supervised or reinforcement learning. Machine learning models may be trained using supervised approaches and/or unsupervised approaches.

At 220, the process may generate an alert in response to the detected movement of the guidewire that devices from the baseline movement and/or from the target position. Exemplary alerts include: a pop-up window presented on a display, an overlay over the guidewire indicating the detected movement, a sound played over speakers (e.g., beep, audio message), and/or a haptic feedback (e.g., vibration of a tool being held by the operator).

Optionally, the detected movement includes a detected displacement of the guidewire in a certain direction, and/or a detected deformation of the shape of the guidewire. The generated overlay for presentation on a display over at least one of the successive images may visually indicate the direction of the displacement of the guidewire (e.g., by an arrow pointing in the direction of the displacement), and/or visually indicating a portion of the guidewire undergoing the deformation (e.g., color coding, bolding, and/or marking the deformed portion of the guidewire).

At 220, action may be taken in response to the detected movement. The action may be automatically implemented by code (e.g., by a robot and/or automated tool performing the procedure and/or assisting with the procedure) and/or by a human such as the operator performing the procedure. The action may be restoring the guidewire to the baseline movement and/or target position. Alternatively, the action may be resetting the system to indicate a new baseline movement and/or a new target position, such as when the movement of the guidewire is a desired movement performed by the operator.

Referring now back to FIG. 3, image 302 may be a fluoroscopy image captured during a medical procedure, in particular, a deployment of an expandable replacement aortic valve 310 over a guidewire 306, e.g., SAFARI guidewire. Guidewire 306 is placed in a left ventricle of a heart of a subject, for use as an anchor. A portion 308 of guidewire 306 within the left ventricle may be automatically detected (e.g., segmented), as described herein. Boundary box 304 indicates a baseline movement of portion 308 of a guidewire 306, delineating the maximal movement of portion 308 occurring during multiple cardiac and/or breathing cycles, when a pose of the image sensor is fixed. Successive images are monitored for detecting movement of portion 308 deviating out of boundary box 304. Such deviation in movement may indicate, for example, erroneous displacement of the guidewire which may lead to damage to the conduction system of the heart. An alert may be detected when portion 308 of guidewire 306 reaches boundary box 304, for preventing the erroneous displacement and reducing or preventing damage to the conduction system of the heart.

Referring now back to FIG. 4, the features of the method described with reference to FIG. 4 may be combined with, include, and/or replace one or more features of the methods described with reference to FIG. 2.

At 402, the processor obtains a target position of the guidewire.

The target position may be obtained from an anatomical image captured prior to the medical procedure, for example, a pre-procedure CT scan. The anatomical image from which the target position is computed may be a still image, optionally a single image, captured during a certain (e.g., random) phase of the heartbeats and/or breathing cycle. Alternatively or additionally, the target position is obtained from one of the baseline images, which may be captured during a certain (e.g., random) phase of the heartbeats and/or breathing cycle.

The anatomical image and/or baseline image from which the target position is obtained may exclude the presence of the guidewire.

The target position may be defined in 2D and/or 3D.

The target position may be defined, for example, as a bounding box and/or non-uniform region that delineates a boundary defining the target location for the guidewire. For example, the interior of the left ventricle, and/or bounding box 304 described with reference to FIG. 3.

The target position may be automatically defined by an ML model that receives the anatomical image as input and outputs the boundary. The ML model may be trained on a training dataset of multiple images depicting the body cavity within which the guidewire will be placed, with ground truth marking of boundaries.

The term baseline movement used herein may include the target position.

At 404, the processor may analyze images of the guidewire, for detecting a current position of the guidewire. The images are captured over multiple heartbeats and/or breathing cycles. The images may be the successive images described herein.

The analysis may be performed by segmenting the guidewire from one or more of the images, for example, using segmentation code and/or a trained ML model, as described herein.

At 406, the processor may determine whether the current position of the guidewire deviates from the target position. For example, whether the guidewire shape has deformed, and/or whether the guidewire has been displaced away from the target position.

When the target position is a boundary region, the determination may be done by correlating the target position of the anatomical image to the images captured over the heartbeats and/or breathing cycles that depict the guidewire. The correlation may be done by correlation code that correlates the anatomical image (e.g., from the pre-procedure CT scan) to the current successive image (e.g., fluoroscopy image). A transformation matrix may be computed according to the correlation. The boundary region defining the target position computed for the anatomical image may be transformed to the successive image using the transformation matrix. The guidewire may be segmented with the successive image, to determine whether the guidewire is within the bounds of the transformed boundary or external to it.

Alternatively or additionally, the target position represents an approximation, and is fixed in shape. The same target position is used for the successive images, to determine deviation.

Alternatively or additionally, the target position which may represent a boundary, is applied to an initial successive image at a certain phase of the heartbeat and/or breathing cycle. A transformation (e.g., matrix) may be computed from the current successive image to a subsequent successive image at another phase of the heartbeat and/or breathing cycle. The transformation is applied to the boundary representing the target position, which is applied to the subsequent successive image, to determine whether the guidewire is within the transformed boundary or not. The transformations dynamically adapt the boundary representing the target position to the changing anatomy, for example, to the shape of the interior of the left ventricle as the left ventricle contracts and expands, and/or as the subject inhales and exhales.

At 408, an alert may be generated in response to the deviation, for example, as described with reference to 220 of FIG. 2.

At 410, action may be taken, for example, as described with reference to 222 of FIG. 2.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant images and guidewires will be developed and the scope of the terms image and guidewire are intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer implemented method of monitoring a guidewire position during a medical procedure, comprising:
analyzing a plurality of baseline images captured over a plurality of at least one of heartbeats and breathing cycles, the plurality of baseline images depicting a guidewire in a body cavity;
computing according to the analysis, a baseline movement of the guidewire during the at least one of heartbeats and breathing cycles; and
monitoring successive images of the guidewire for detecting movement of the guidewire that deviates from the baseline movement.

2. The computer implemented method of claim 1, wherein analyzing comprises computing movement of the guidewire within the plurality of baseline images captured by an image sensor at a fixed pose, wherein the baseline movement of the guidewire comprises maximum ranges of movement of the guidewire during the at least one of heartbeats and breathing cycles, and wherein movement of the guidewire is detected when movement of the guidewire depicted in the successive images deviates from the baseline movement comprising the maximum ranges of movement.

3. The computer implemented method of claim 2, further comprising automatically detecting the fixed pose.

4. The computer implemented method of claim 1, wherein analyzing comprises identifying at least one deformation of the guidewire within the plurality of baseline images captured by an image sensor at a fixed pose, wherein the baseline movement of the guidewire comprises the identified at least one deformation during the at least one of heartbeats and breathing cycles, and wherein movement of the guidewire is detected when deviation of the guidewire depicted in the successive images deviates from the at least one deformation of the baseline movement.

5. The computer implemented method of claim 1, wherein analyzing comprises computing shape and/or movement of the guidewire relative to at least one fiducial marker that remains in a fixed location during the plurality of at least one of heart beats and breathing cycles, wherein the baseline movement of the guidewire is a range of shapes and/or movements relative to the at least one fiducial marker, wherein the movement of the guidewire that deviates from the baseline movement is determined by analyzing the shape and/or movement of the guidewire relative to the at least one fiducial marker.

6. The computer implemented method of claim 5, wherein the at least one fiducial marker is selected from a group comprising: at least one anatomical structure of the subject, and an object affixed on the subject.

7. The computer implemented method of claim 1, wherein analyzing comprises assigning a label to each of the plurality of baseline images indicating a phase during at least one of: a heartbeat cycle, and a breathing cycle, wherein monitoring comprises assigning the label to each successive image, and wherein detecting movement comprises determining a deviation of a location of the guidewire in each successive image of a certain label from the location of the guidewire in a baseline image with a label matching the certain label.

8. The computer implemented method of claim 1, wherein analyzing comprises fitting a polynomial with a plurality of parameters to the guidewire depicted in the plurality of baseline images, wherein the plurality of parameters are adapted for fitting the polynomial to the guidewire depicted in each respective baseline image, wherein the baseline movement comprises at least one range of values of the plurality of parameters of the polynomial, wherein monitoring comprises computing values for the plurality of parameters for fitting the polynomial to the guidewire depicted in the successive images, and movement of the guidewire that deviates from the baseline movement is detected when the plurality of parameters are different than the at least one range.

9. The computer implemented method of claim 1, wherein analyzing comprises fitting a spline comprising a plurality of piecewise curves to the guidewire depicted in the plurality of baseline images, wherein the piecewise curves are adapted for fitting the spline to the guidewire depicted in each respective baseline image, wherein the baseline movement comprises variations of each of the plurality of piecewise curves of the spline, wherein monitoring comprises fitting the spine to the guidewire depicted in the successive images, and movement of the guidewire that deviates from the baseline movement is detected by analyzing each of the plurality of piecewise curves of the spline to detect deviation of a certain piecewise curve from the baseline movement.

10. The computer implemented method of claim 1, further comprising creating at least one training record that includes at least one baseline image of the plurality of baseline images and a ground truth label indicating the baseline movement of the guidewire, training a machine learning model on the at least one training record, wherein monitoring comprises feeding the successive images into the machine learning model, and wherein detecting movement of the guidewire that deviates from the baseline moment is obtained as an outcome of the machine learning model.

11. The computer implemented method of claim 10, wherein the at least one training record includes a sequence of the plurality of baseline images captured over a plurality of at least one of heartbeats and breathing cycles, wherein feeding comprises feeding a plurality of successive images captured over a plurality of at least one of heartbeats and breathing cycles.

12. The computer implemented method of claim 1, wherein detecting movement comprises detecting at least one of: displacement of the guidewire in a certain direction, and at least one deformation of the guidewire, and further comprising generating an overlay for presentation on a display over at least one of the successive images indicating the at least one of: the certain direction of the displacement of the guidewire, and a portion of the guidewire undergoing the at least one deformation.

13. The computer implemented method of claim 1, further comprising analyzing the successive images to detect a change in pose of an image sensor that captures the images, and in response to the detected change, computing a new baseline movement of the guidewire from images captured after the change in pose is detected.

14. The computer implemented method of claim 1, further comprising analyzing the successive images to detect a change in pose of an image sensor that captures the images, and in response to the detected change, computing a transformation of a current position of the guidewire according to a transformation from a current pose of the image sensor to a preceding pose of the image sensor, and wherein the detecting movement of the guidewire that deviates from the baseline movement is according to the transformation of the current position of the guidewire.

15. The computed implemented method of claim 1, further comprising segmenting the guidewire from the plurality of images and of the successive images, wherein the analysis and the monitoring is according to the segmented guidewire.

16. The computer implemented method of claim 1, further comprising identifying tissues in proximity to the guidewire that are likely to be damaged by movement of the guidewire that deviates from the baseline movement, and wherein at least one of the analyzing, the computing the baseline movement, the monitoring, and the detecting movement, is for a portion of the guidewire in proximity to the identified tissues and is not performed for another portion of the guidewire that is not in proximity to the identified tissues.

17. The computer implemented method of claim 1, wherein the body cavity comprises a left ventricle of the heart, wherein the guidewire is deformed and/or is moved within the left ventricle.

18. The computer implemented method of claim 1, wherein the plurality of baseline images depict the guidewire at a target position, and wherein monitoring comprises monitoring the successive images of the guidewire for detecting movement of the guidewire from the target position that deviates from the baseline movement.

19. The computer implemented method of claim 1, further comprising generating an alert in response to detecting movement of the guidewire that deviates from the baseline movement.

20. A system for monitoring a guidewire during a medical procedure, comprising:
at least one processor executing a code for:
analyzing a plurality of baseline images captured over a plurality of at least one of heartbeats and breathing cycles, the plurality of baseline images, depicting a guidewire in a body cavity;
computing according to the analysis, a baseline movement of the guidewire during the at least one of heartbeats and breathing cycles; and
monitoring successive images of the guidewire for detecting movement of the guidewire that deviates from the baseline movement.
